(19) United States
(12) Patent Application Publication
(10) Pub. No.: US 2019/0162566 A1
(43) Pub. Date: May 30, 2019

(54) SYSTEM AND METHOD FOR TRACKING LIQUID CONSUMPTION

(71) Applicant: Groking Lab Limited, Kowloon (HK)

(72) Inventors: Sze Ring PAU, Kowloon (HK); Wai Ho TSANG, Kowloon (HK); Chung Ming TONG, Kowloon (HK)

(21) Appl. No.: 15/825,284

(22) Filed: Nov. 29, 2017

(51) Int. Cl. G01F 1/075; A61B 5/00

(52) U.S. Cl. CPC G01F 1/0755; G01F 15/063; A61B 5/4875

(57) ABSTRACT

A method for tracking liquid consumption includes determining, using a portable apparatus for monitoring liquid consumption, data indicative of an amount of liquid consumed by a user from a liquid container. The method also includes transmitting the determined data to an activity tracking device operably connected with the portable apparatus, for determination of: an amount of liquid remaining in the liquid container; and a liquid consumption plan based on the amount of liquid remaining, activity data, and user activity data arranged in the activity tracking device.

### TECHNICAL FIELD

The invention relates to a system and method for tracking liquid consumption. The invention also relates to a portable apparatus for monitoring liquid consumption, and in particular, a flow meter operable to communicate with an external electric device for monitoring beverage consumption.

### BACKGROUND

Water plays an important physiological role in the survival of human beings as a large portion of the human body, in terms of weight or volume, is made up of water. In order for the body to properly grow, develop, and function, proper fluid (liquid) balance in the body is essential. Depending on age, gender, weight, height, and other body conditions, the body water requirement may vary for different people.

The human body has the intrinsic ability to regulate water content in different tissues and organs of different body parts, and it can produce a thirst sensation to remind one of the need to consume liquid to stay properly hydrated when the water content of the body falls below a threshold as detected by the brain. Problematically, however, in the nowadays rapidly paced society, people are busy with a lot of different tasks and work, and they often neglect the signals generated by their body.

Conventional ways of keeping track of one's fluid consumption behaviour is by pen and paper. Whenever a fluid consumption event occurs, a note is written on the paper detailing the amount of fluid consumed. More recently, applications have been developed to allow users to record their fluid consumption events, digitally on portable electronic devices. These applications also allow users to review consumption history.

These known ways of keeping track of one's fluid consumption behaviour is inefficient and unreliable. In one example, the user may forget to record (write down or input in application) a fluid consumption event. In some examples, even if the user remembers to record, he/she may not know accurately how much liquid is consumed, for example due to the lack of marker on the beverage container to indicate the amount of liquid consumed. In another example, when one is involved in intense exercise such as hiking and cycling in outdoor settings, the fluid consumption event(s) cannot be properly recorded.

### SUMMARY OF THE INVENTION

In accordance with a first aspect of the invention, there is provided a method for tracking liquid consumption, comprising: determining, using a portable apparatus for monitoring liquid consumption, data indicative of an amount of liquid consumed by a user from a liquid container; and transmitting the determined data to an activity tracking device operably connected with the portable apparatus, for determination of at least one of: an amount of liquid remaining in the liquid container; and a liquid consumption plan based on the amount of liquid remaining in the liquid container, activity data, and user activity data arranged in the activity tracking device.

In one embodiment of the first aspect, the activity data comprises at least one of: a type of activity being performed or to be performed by the user; a predetermined duration of the activity being performed or to be performed by the user; a completion state of the activity; geographic data of the environment in which the activity is being or is to be performed; and weather condition of an environment in which the activity is being or is to be performed.

In one embodiment of the first aspect, the weather condition comprises at least one of: humidity level of the environment; temperature level of the environment; and UV index of the environment.

In one embodiment of the first aspect, the geographic data includes at least one of: latitude, longitude and altitude of the environment.

In one embodiment of the first aspect, the liquid consumption plan is determined further based on a liquid consumption record of the user during the activity.

In one embodiment of the first aspect, the user activity data comprises physiological data of the user during the activity.

In one embodiment of the first aspect, the physiological data includes at least one of: heart rate, pulse rate, and blood oxygen level of the user.

In one embodiment of the first aspect, determination of a liquid consumption plan comprises updating an existing liquid consumption plan.

In one embodiment of the first aspect, the liquid consumption plan is determined further based on a liquid consumption goal set by the user.

In one embodiment of the first aspect, the method also includes storing, at the activity tracking device, data of an initial amount of liquid in the liquid container.

In one embodiment of the first aspect, the liquid consumption plan comprises at least one liquid consumption reminder to be provided to the user at a specific time.

In one embodiment of the first aspect, the liquid consumption reminder comprises an amount of liquid to be consumed.

In one embodiment of the first aspect, the method also includes determining, at the activity tracking device, at least one of: an amount of liquid remaining in the liquid container; and a liquid consumption plan based on an amount of liquid remaining in the liquid container, activity data, and user activity data arranged in the activity tracking device.

In one embodiment of the first aspect, the portable apparatus is arranged to communicate directly and wirelessly with the activity tracking device.

In one embodiment of the first aspect, the method further comprises transmitting the determined data to an electronic device operably connected with the portable apparatus for record or analysis.

In one embodiment of the first aspect, the electronic device is arranged to receive, from the activity tracking device, at least one of: liquid consumption plan, liquid consumption record, activity data, and user activity data.

In one embodiment of the first aspect, the electronic device is arranged to analyze data received from the activity tracking device and from the portable apparatus.

In one embodiment of the first aspect, the electronic device is arranged to use the analysed data for determination of future liquid consumption plan.

In one embodiment of the first aspect, the electronic device comprises a mobile phone, a tablet, or a computer.

In one embodiment of the first aspect, the portable apparatus comprises: an inlet arranged to receive liquid; an outlet arranged to output the received liquid; a channel enabling fluid communication between the inlet and the outlet; a detector arranged to detect m one or more flow properties of liquid passing in the channel for determination of an amount of liquid consumed by a user from a liquid container; and a communication module operably connected with the detector for communicating data indicative of the amount of liquid consumed to an external electronic device.

In accordance with a second aspect of the invention, there is provided a system for tracking liquid consumption, comprising: a portable apparatus for monitoring liquid consumption, arranged to determine an amount of liquid consumed by a user from a liquid container; transmit the determined data to an activity tracking device operably connected with the portable apparatus, for determination of: an amount of liquid remaining in the liquid container; and a liquid consumption plan based on the amount of liquid remaining, activity data, and user activity data arranged in the activity tracking device.

In one embodiment of the second aspect, the activity data comprises at least one of: a type of activity being performed or to be performed by the user; a predetermined duration of the activity being performed or to be performed by the user; a completion state of the activity; geographic data of the environment in which the activity is being or is to be performed; and weather condition of an environment in which the activity is being or is to be performed.

In one embodiment of the second aspect, the weather condition comprises at least one of: humidity level of the environment; temperature level of the environment; and UV index of the environment.

In one embodiment of the second aspect, the geographic data includes at least one of: latitude, longitude and altitude of the environment.

In one embodiment of the second aspect, the liquid consumption plan is determined further based on a liquid consumption record of the user during the activity.

In one embodiment of the second aspect, the user activity data comprises physiological data of the user during the activity.

In one embodiment of the second aspect, the physiological data includes at least one of: heart rate, pulse rate, and blood oxygen level of the user.

In one embodiment of the second aspect, determination of a liquid consumption plan comprises updating an existing liquid consumption plan.

In one embodiment of the second aspect, the liquid consumption plan is determined further based on a liquid consumption goal set by the user.

In one embodiment of the second aspect, the activity tracking device is arranged to store data of an initial amount of liquid in the liquid container.

In one embodiment of the second aspect, the liquid consumption plan comprises at least one liquid consumption reminder to be provided to the user at a specific time.

In one embodiment of the second aspect, the liquid consumption reminder comprises an amount of liquid to be consumed.

In one embodiment of the second aspect, the system also includes the activity tracking device arranged to determine: an amount of liquid remaining in the liquid container; and a liquid consumption plan based on the amount of liquid remaining, activity data, and user activity data arranged in the activity tracking device.

In one embodiment of the second aspect, the portable apparatus for monitoring liquid consumption is arranged to communicate directly and wirelessly with the activity tracking device.

In one embodiment of the second aspect, the portable apparatus is arranged to transmit the determined data to an electronic device operably connected with the portable apparatus.

In one embodiment of the second aspect, the system further includes the electronic device, wherein the electronic device is arranged to receive, from the activity tracking device, at least one of: liquid consumption plan, liquid consumption record, activity data, and user activity data.

In one embodiment of the second aspect, the electronic device is arranged to analyze data received from the activity tracking device and from the portable apparatus.

In one embodiment of the second aspect, the electronic device is arranged to use the analysed data for determination of future liquid consumption plan.

In one embodiment of the second aspect, the electronic device comprises a mobile phone, a tablet, or a computer.

In one embodiment of the second aspect, the portable apparatus comprises: an inlet arranged to receive liquid; an outlet arranged to output the received liquid; a channel enabling fluid communication between the inlet and the outlet; a detector arranged to detect one or more flow properties of liquid passing in the channel for determination of an amount of liquid consumed by a user from a liquid container; and a communication module operably connected with the detector for communicating data indicative of the amount of liquid consumed to an external electronic device.

In accordance with a third aspect of the invention, there is provided a portable apparatus for monitoring liquid consumption, comprising an inlet arranged to receive liquid, an outlet arranged to output the received liquid, a channel enabling fluid communication between the inlet and the outlet, a detector arranged to detect one or more flow properties of liquid passing in the channel, and a communication module operably connected with the detector for communicating the one or more detected flow properties to an external electronic device for monitoring liquid consumption. The liquid is preferably a beverage, e.g., water. The portable apparatus in this aspect may be used as the portable apparatus of the first aspect.

In one embodiment of the third aspect, the portable apparatus further comprises a quick release coupler arranged at the inlet for connection and disconnection with a liquid source. The liquid source may comprise a container and a tube for connection with the inlet.

In one embodiment of the third aspect, the quick release coupler comprises an actuator arranged to be actuated by a user for ready-connection and disconnection of the liquid source.

In one embodiment of the third aspect, the detector comprises a flow meter arranged to detect a flow rate or flow volume of liquid in the channel. Preferably, the flow meter is suction-activated.

In one embodiment of the third aspect, the portable apparatus further comprises a valve arranged to prevent backflow of liquid in the channel. The valve may be arranged downstream or upstream of the flow meter. In some embodiments, multiples valves may be used, arranged both downstream and upstream of the flow meter.

In one embodiment of the third aspect, the flow meter comprises an axial flow turbine arranged in the channel and a sensor arranged to monitor movement of the axial flow turbine. Preferably, the movement is rotation. The flow meter may determine the flow rate or flow volume based on the detected movement of the turbine.

In one embodiment of the third aspect, the axial flow turbine comprises a plurality of vanes, at least one of the vanes includes a magnetic member, and the sensor comprises a magnetic sensor for detecting movement of the magnetic member. Preferably, the magnetic sensor is arranged to detect rotation of the magnetic member as the turbine rotates. The magnetic sensor may be a Hall sensor.

In one embodiment of the third aspect, the portable apparatus further comprises a power source arranged to power electrical components of the portable apparatus. These electrical components may include the detector and the communication module. These electrical components may further include one or more of: a processor, a memory/storage, a clock, a display, etc.

In one embodiment of the third aspect, the power source is rechargeable.

In one embodiment of the third aspect, the portable apparatus further comprises a charging port arranged for charging a rechargeable power source in the portable apparatus.

In one embodiment of the third aspect, the communication module comprises a wireless communication module. Preferably, the wireless communication module comprises one or more of: a Bluetooth communication module arranged to communicate with an external electronic device using a Bluetooth communication link; and an ANT+ communication module arranged to communicate with an external electronic device using an ANT+ communication link. In other embodiments, the wireless communication module may alternatively or additionally comprises one or more communication modules that utilize LTE, Wi-Fi, NFC, ZigBee, etc. communication links.

In one embodiment of the third aspect, the portable apparatus further comprises a clock for determining a time stamp associated with the one or more detected flow properties. Preferably, the communication module is further arranged to communicate the time stamp associated with the one or more detected flow properties to an external electronic device.

In one embodiment of the first aspect, the portable apparatus further comprises a mouthpiece arranged at the outlet. The mouthpiece is preferably elastic and may comprise a bite valve.

In one embodiment of the first aspect, the mouthpiece is detachably connected at the outlet.

In one embodiment of the third aspect, the portable apparatus further comprises a processor arranged to process the one or more detected flow properties. The processor may be operable to execute program instructions that allow the portable apparatus to interact with the user. For example, the processor may include program instructions for operating the portable apparatus in different modes, or for selectively activating and deactivating different functional modules based on user input.

In one embodiment of the third aspect, the portable apparatus further comprises input means for enabling a user to provide input to the processor. The input means may be actuators, buttons, dials, touch sensitive display, etc.

In one embodiment of the third aspect, the portable apparatus further comprises a storage operably connected with the processor and arranged to store the one or more detected flow properties. The storage may comprise one or both of volatile and non-volatile memory.

In one embodiment of the third aspect, the portable apparatus further comprises a display operably connected with the processor and arranged to display information related to the one or more flow properties or to liquid consumption. The display may also display the time stamp associated with the one or more flow properties.

In one embodiment of the third aspect, the portable apparatus further comprises an inertial measurement unit operably connected with the processor and arranged to detect an orientation of the portable apparatus; and the processor is arranged to control an orientation of information provided on the display based on a detected orientation of the apparatus.

In one embodiment of the third aspect, the information related to liquid consumption includes one or more of: an aggregate amount of liquid consumed; an amount of liquid consumed in a previous liquid consumption event; and a percentage representing a ratio of liquid consumed to a targeted liquid consumption.

In accordance with a fourth aspect of the invention, there is provided a system for monitoring liquid consumption, comprising: a portable apparatus; and an external electronic device arranged to receive the one or more detected flow properties from the portable apparatus; wherein the external electronic device is arranged to determine, based on the one or more detected flow properties, an amount of liquid consumed, an aggregate amount of liquid consumed and provide an alarm to a user when the determined aggregate amount of liquid consumed is less than an expected quantity of liquid consumption.

In a preferred embodiment of the fourth aspect, the portable apparatus is the portable apparatus of the third aspect.

In one embodiment of the fourth aspect, the alarm may be in the form of one or more of: a visual alarm, an audible alarm, and a tactile alarm.

In one embodiment of the fourth aspect, the external electronic device comprises any of a mobile phone, a computer, a smart watch, and a portable health tracker device.

In accordance with a fifth aspect of the invention, there is provided a portable hydration kit comprises a portable liquid source; and a portable apparatus for monitoring liquid consumption, arranged for connection with the portable liquid source.

In a preferred embodiment of the fifth aspect, the portable apparatus is the portable apparatus of the third aspect.

In one embodiment of the fifth aspect, the portable liquid source includes a hydration bladder and a connection tube.

In accordance with a sixth aspect of the invention, there is provided a method for monitoring liquid consumption. The method comprises detecting, using a portable apparatus for monitoring liquid consumption, one or more flow properties of liquid passing in the channel of the portable apparatus; and communicating the one or more detected flow properties to an external electronic device for monitoring liquid consumption.

In a preferred embodiment of the sixth aspect, the portable apparatus is the portable apparatus of the third aspect.

In one embodiment of the sixth aspect, the method further comprises the steps of: determining, at the external electronic device, an amount of liquid consumed based on the one or more detected flow properties; determining an aggregate amount of liquid consumed; and providing an alarm to a user when the determined aggregate amount of liquid consumed is less than an expected quantity of liquid consumption.

In one embodiment of the sixth aspect, the alarm may be in the form of one or more of: a visual alarm, an audible alarm, and a tactile alarm.

In accordance with a seventh aspect of the invention, there is provided a method for tracking liquid consumption, comprising: receiving, from a portable apparatus for monitoring liquid consumption from a liquid container, data indicative of an amount of liquid consumed by a user from the liquid container; and determining, based on the data received, an amount of liquid remaining in the liquid container. Preferably, the method is performed at an activity tracking device, such as but not limited to that in the above aspects of the invention.

In one embodiment of the seventh aspect, the method further comprises determining, based on the received data, activity data, and user activity data, a liquid consumption plan.

In one embodiment of the seventh aspect, the activity data comprises at least one of: a type of activity being performed or to be performed by the user; a predetermined duration of the activity being performed or to be performed by the user; a completion state of the activity; geographic data of the environment in which the activity is being or is to be performed; and weather condition of an environment in which the activity is being or is to be performed.

In one embodiment of the seventh aspect, the user activity data comprises physiological data of the user during the activity.

In one embodiment of the seventh aspect, the physiological data includes at least one of: heart rate, pulse rate, and blood oxygen level of the user.

In one embodiment of the seventh aspect, the liquid consumption plan is determined further based on a liquid consumption record of the user during an activity.

In one embodiment of the seventh aspect, determination of a liquid consumption plan comprises updating an existing liquid consumption plan.

In one embodiment of the seventh aspect, the liquid consumption plan is determined further based on a liquid consumption goal set by the user.

In one embodiment of the seventh aspect, the method further comprises storing data of an initial amount of liquid in the liquid container.

In one embodiment of the seventh aspect, the liquid consumption plan comprises at least one liquid consumption reminder to be provided to the user at a specific time.

In one embodiment of the seventh aspect, the liquid consumption reminder comprises an amount of liquid to be consumed.

### BRIEF DESCRIPTION OF THE DRAWINGS

Embodiments of the invention will now be described, by way of example, with reference to the accompanying drawings in which:

FIG. 1A is a side view of a portable apparatus for monitoring liquid consumption in accordance with one embodiment of the invention;

FIG. 1B is another side view of the portable apparatus of FIG. 1A;

FIG. 1C is a bottom view of the portable apparatus of FIG. 1A;

FIG. 1D is a top view of the portable apparatus of FIG. 1A;

FIG. 1E is a cross-sectional view of the portable apparatus of FIG. 1A, taken along line A-A in FIG. 1B;

FIG. 1F is an exploded view of the portable apparatus of FIG. 1A;

FIG. 2 is a functional block diagram of a portable apparatus for monitoring liquid consumption in accordance with one embodiment of the invention;

FIG. 3 is a schematic diagram illustrating a liquid consumption monitoring system in accordance with one embodiment of the invention;

FIG. 4 is a flow diagram illustrating the general operation of the liquid consumption monitoring system of FIG. 3 in accordance with one embodiment of the invention; and

FIG. 5 is a flow diagram illustrating an operation of the liquid consumption monitoring system of FIG. 3 in accordance with one embodiment of the invention.

FIG. 6 is a schematic diagram illustrating a system for tracking liquid consumption in accordance with one embodiment of the invention;

FIG. 7A is an example software application interface (showing an activity and liquid consumption record) arranged to be displayed on an electronic device;

FIG. 7B is an example interface of a social network platform for sharing activity information arranged to be displayed on an electronic device;

FIG. 7C is an example interface showing the activity and liquid consumption record on the social network platform arranged to be displayed on an electronic device;

FIG. 8A is an example of a screen interface of an activity tracking device displaying a completion percentage of liquid consumption goal arranged to be displayed on the smart watch of FIG. 6;

FIG. 8B is an example of a screen interface of an activity tracking device displaying an initial amount of liquid in the liquid container of the portable apparatus, arranged to be displayed on the smart watch of FIG. 6;

FIG. 8C is an example of a screen interface of an activity tracking device for users to set the initial amount of liquid in the liquid container of the portable apparatus, arranged to be displayed on the smart watch of FIG. 6;

FIG. 8D is an example of a screen interface of an activity tracking device displaying notification to the user, arranged to be displayed on the smart watch of FIG. 6;

FIG. 8E is an example of a screen interface of an activity tracking device displaying four pieces of information relating to activity and liquid consumption level, arranged to be displayed on the smart watch of FIG. 6; and

FIG. 8F is an example of a screen interface of an activity tracking device displaying a summary of activity and liquid consumption level, arranged to be displayed on the smart watch of FIG. 6.

### DETAILED DESCRIPTION OF THE PREFERRED EMBODIMENT

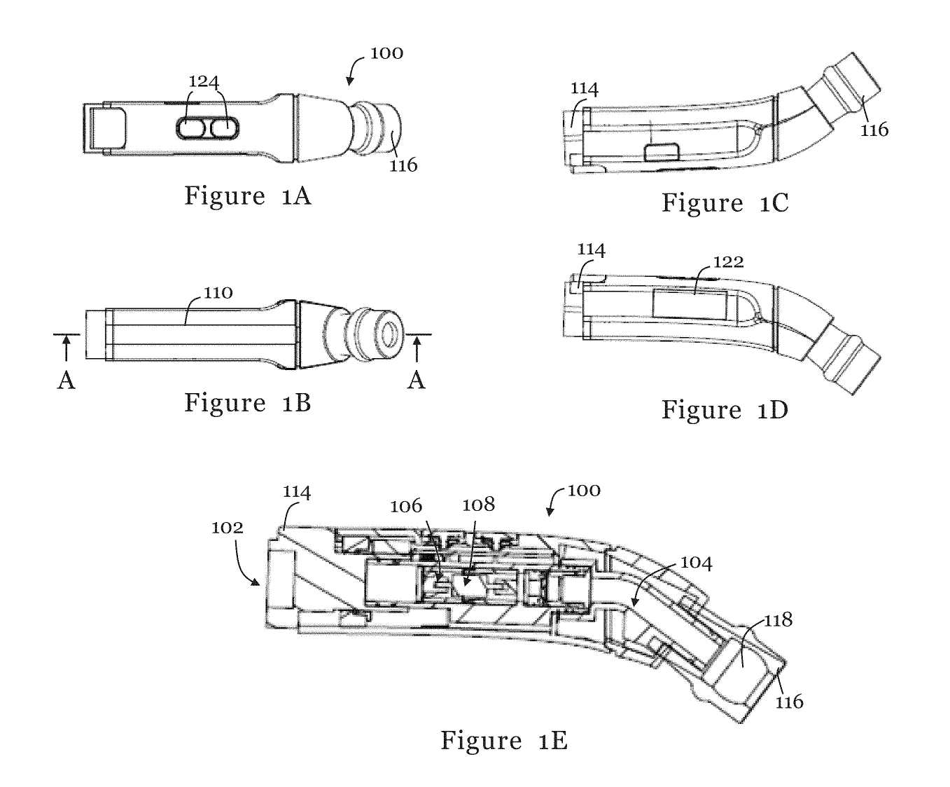

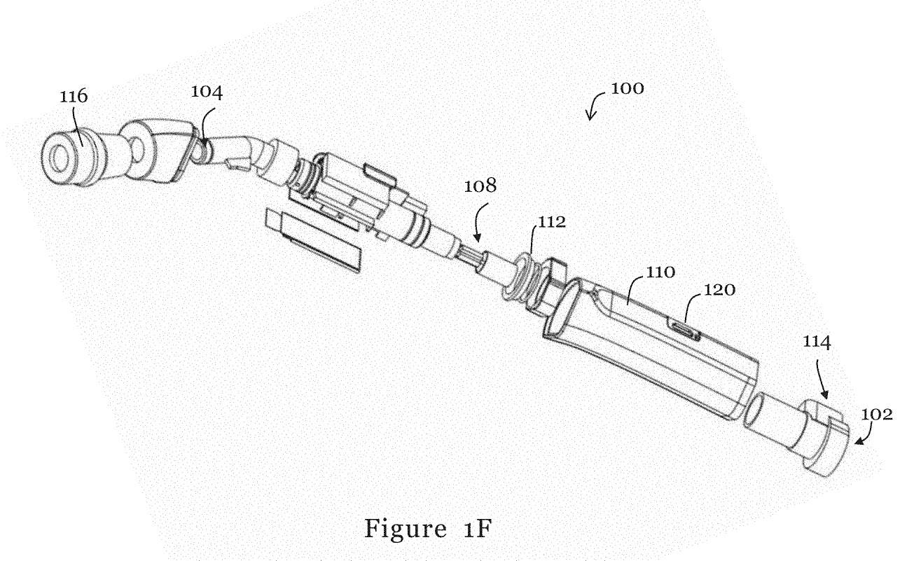

FIGS. 1A to 1F illustrate a portable apparatus 100 for monitoring liquid consumption. The apparatus 100 includes an inlet 102 arranged to receive liquid from a liquid source and an outlet 104 arranged to output the received liquid. A channel 106 is arranged between the inlet 102 and the outlet 104 for enabling fluid flow between the inlet 102 and the outlet 104. The apparatus 100 also includes a detector 108 arranged to detect one or more flow properties of liquid passing in the channel 106, and a communication module operably connected with the detector for communicating the one or more detected flow properties to an external electronic device for monitoring liquid consumption. The external electronic device can be a mobile phone, a computer, a tablet, a smart watch, a portable health tracker device, etc. The liquid is preferably a beverage, for example, water.

As shown in FIGS. 1A-1F, the portable apparatus 100 includes a housing 110. The housing 110 is generally elongated along an axis, with the inlet 102 at one end and the outlet 104 at the other end, and the outlet end of the housing 110 being bent to one side. In this embodiment, the channel 106 in the housing 110 generally extends along the housing axis. The housing 110 may be made of relatively strong but light-weighted material, such as plastic or carbon fibre, so that the apparatus 100 is substantially shockproof and be easily carried by a user. One or more seal rings 112 may be positioned in the housing 110 for providing water-tight sealing. In this embodiment, a seal ring 112 is placed at the inlet for preventing leakage of liquid upon connection of liquid source at the inlet 102. In the present embodiment, the housing 110 is arranged such that various electronic components of the apparatus 100 are isolated from the channel 106 in which liquid passes for protection against water and preferably also dust.

In a preferred embodiment, a quick release coupler 114 is arranged at the inlet 102 for connection and disconnection with a liquid source, or more particularly with a connector or tube of a liquid source. The coupler 114 includes a locking mechanism and an actuator that can be actuated by a user to lock and unlock the locking mechanism. The coupler 114 allows ready connection and disconnection of the liquid source from the inlet 102. In the present embodiment, the actuator is a push actuator movable in a direction generally perpendicular to the housing axis. When the actuator is in the normal biased position, the locking mechanism, if connected with a connector of the liquid source, is arranged to firmly engage the connector of the liquid source. The engagement is preferably also substantially fluid tight. To connect the connector of the liquid source to the inlet 102, a user can actuate the actuator along an actuator axis and connect the tube or connector to the inlet in a direction generally perpendicular to the actuator axis. The user can then release the actuator to lock the locking mechanism such that the tube or connector is firmly secured by the locking mechanism. In one embodiment, the coupler 114 is arranged such that the connection motion of the tube or connector along the direction generally perpendicular to the actuator axis can create a sufficient force to overcome the biasing force of the actuator. In such case, the user need not actuate the actuator for connection. To remove the connector of the liquid source from the inlet 102, the user has to actuate the actuator to overcome the biasing force. This readily releases the coupling. In the present embodiment, the actuator is a push button, but in other embodiments, the actuator may be a dial, a toggle switch, a slide switch, a rotary button, etc. In some embodiments, the coupler 114 may include a threaded coupling, a push-fit coupling, or a bayonet coupling.

Preferably, a mouthpiece 116 is arranged at the curved outlet end of the housing 110. The mouthpiece 116 defines a channel 118 that is continuous with the channel 106. Preferably, the mouthpiece 116 can be detachably connected to the housing 110 through a fitting mechanism (snap fit, friction fit, etc.) for replacement, repair, cleaning, etc. when needed. The coupling between the mouthpiece 116 and the housing 110 is preferably substantially fluid tight. The mouthpiece 116 is preferably elastic and it may include a bite valve. In this embodiment, the mouthpiece 116 extends generally along the deviated direction of the outlet end of the housing 110. In operation, the user applies suction at the mouthpiece 116 to draw liquid from the liquid source to the inlet 102 through the channel 106 and hence out of the mouthpiece 116, for consuming the liquid.

The detector 108 for detecting flow properties of liquid passing in the channel 106 is arranged inside the housing 110. Preferably, the detector 108 comprises a flow meter for detecting flow rate or flow volume of liquid passing in the channel 106. The flow meter may be a suction-activated flow meter. In a preferred embodiment, the flow meter comprises an axial flow turbine arranged in the channel 106, and a sensor arranged to monitor the movement (e.g., rotation) of the axial flow turbine. The axial flow turbine may be mounted on a shaft and include a plurality of vanes. Preferably, the vanes are angled or spiralled such that the axial flow turbine rotates when liquid flows through it. Preferably, at least one of the vanes includes a magnetic member such as a magnet and the sensor includes a corresponding magnetic sensor such as a Hall-effect sensor. The sensor is preferably fixed external to the channel and is operable to generate a pulse when it detects the magnetic member as the turbine rotates. Generally, the faster the turbine rotates, the more frequent the pulses are detected. In some embodiments, the sensor may be operable to determine a direction of rotation of the turbine based on the pattern of the detected pulses. Preferably, the sensor detects the speed of rotation of the turbine or counts the number (or direction) of revolutions of the turbine. This information, along with the time or duration of flow, can then be used for determining the flow rate or flow volume of liquid based on predetermined geometric parameters of the channel 106 and the detector 108.

It should be noted that the detector 108 comprising an axial flow turbine with magnetic vanes and corresponding magnetic sensor in this embodiment are merely exemplary. In some embodiments, the rotation of turbine may be detected mechanically, optically, electrically, etc. In some embodiments, other types of flow meters may be used. For example, the flow meter may be a radial flow turbine with a shaft generally perpendicular to the fluid flow direction.

One or more valves may be arranged in the apparatus 100, for example, in positions upstream of the flow meter, downstream of the flow meter, or both. These valves only permit flow in one direction from the inlet 102 to the outlet 104, and they can prevent backflow of liquid, especially after the user withdraws the suction force at the mouthpiece 116. With these valves, detection errors caused by backflow may be prevented or at least reduced. In an alternative embodiment, a valve may not be necessary as the detector 108 may be able to differentiate between forward and backward flow, and so can correct for the detected flow volume or flow rate accordingly. In one embodiment, the valve may be positioned immediately downstream or upstream of the flow meter such that little gap is arranged between the valve and the flow meter. In another embodiment, one valve is positioned downstream of the flow meter at the end of the mouthpiece 116, one valve is positioned upstream of the flow meter. The valve(s), if appropriately positioned, can minimize leakage of liquid from the mouthpiece 116 after the user withdraws the suction force at the mouthpiece 116.

The portable apparatus 100 may include a power source arranged to power the electrical components of the apparatus 100. Preferably, the power source is rechargeable, and may be a battery with lithium-based chemistry. As shown in FIG. 1F, a charging port 120 is arranged in the housing 110 for allowing the power source to be charged. In one embodiment, the charging port 120 may be a USB port that is optionally covered by a cover when not in use. A USB cable connected to an external power source (e.g., AC power, adapter, etc.) can be plugged into the charging port 120 for charging the power source. In some embodiments, the USB port may be used to transfer data, separately from or in addition to providing power.

A display 122 may be provided on the housing 110 to display information related to the detected flow properties or to the liquid consumption (e.g., instant consumption volume, cumulative consumption volume, targeted consumption volume, etc.). In some embodiments, the display 122 may also display user instruction, time, etc. The display 122 is preferably an LED display. In this embodiment, two buttons 124 are arranged on the housing 110 for allowing the user to control operation of the apparatus 100 and to input information or data into the apparatus 100. In this embodiment, the two buttons are spring-biased push buttons but in other embodiments they may be of different form.

Although not clearly shown in FIGS. 1A-1F, the apparatus 100 may also include one or more of: a processor, a memory, a communication module, a clock, an inertial measurement unit, and an alarm module. In one embodiment, the apparatus 100 may include additional or alternative functional components.

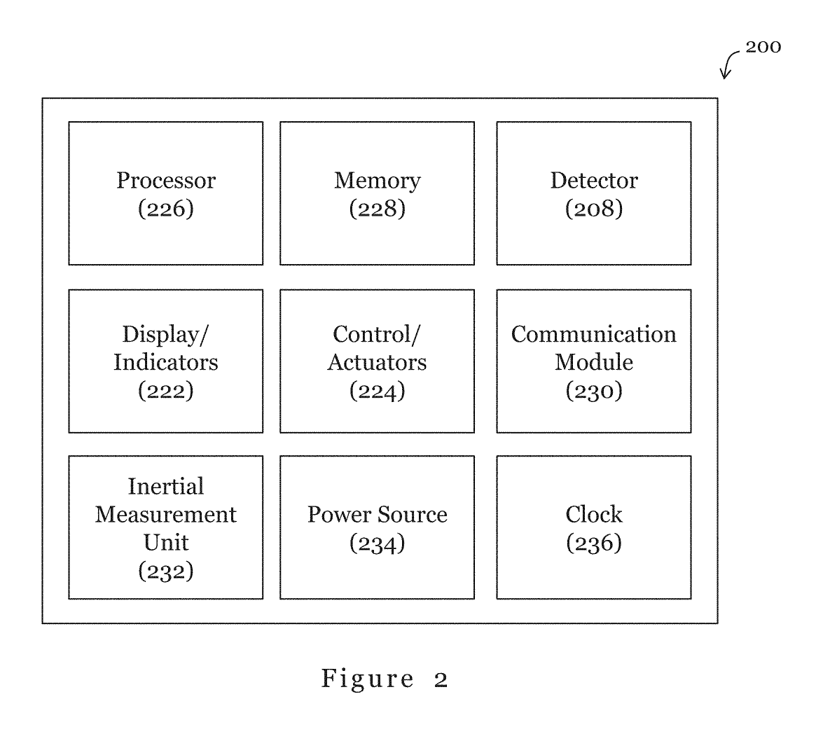

FIG. 2 shows the major functional components of a portable apparatus 200 in one embodiment of the invention. The portable apparatus 200 has similar construction with the apparatus 100 shown in FIGS. 1A-1F. It should be appreciated that the functional modules in FIG. 2 can be implemented using various electrical and/or mechanical components suitably positioned in the apparatus 200. Also, the function modules, where appropriate, are operably connected with each other. Preferably, the functional modules are arranged in the housing of the apparatus 200 such that they are water- and dust-proof.

The portable apparatus 200 includes a processor 226. The processor 226 may be arranged in an electric circuit, and it may be operably connected with the other functional modules. The processor 226 is arranged to process information related to the detected flow properties such as flow rate or flow volume of liquid passing in the channel of the apparatus 200. The processor 226 may also receive a time stamp associated with the detected flow properties from the clock 236.

In one example, the processor 226 may be operable to determine a liquid consumption routine of a user of the portable apparatus 200 based on the detected information. The liquid consumption routine may include a cumulative amount of liquid consumed by the user, the amount of liquid consumed in the present liquid consumption event, the amount of liquid consumed in a previous liquid consumption event, a percentage representing a ratio of liquid consumed to a targeted liquid consumption, as well as time and/or duration associated with these events. The processor 226 may generate a liquid consumption alarm signal based on the liquid consumption routine of the user, and provide the alarm to an alarm module in the apparatus 200 to remind the user of the need to consume liquid at an appropriate time. The alarm module may provide visual alarm, audible alarm, or tactile alarm. In one example, the processor 226 can determine an amount of liquid that the user should consume for a particular day or for a particular activity in order to stay hydrated. The processor 226 may create a liquid consumption alarm based on the liquid consumption history of the user (e.g., the amount of liquid consumed in a certain time period, time since last consumption, etc.), amount of consumption needed to reach targeted liquid consumption and optionally the personal information of the user (e.g., weight, height, and age of the user). The liquid consumption alarm can be provided by appropriate alarm module at a time determined by the processor 226, so as to remind the user to consume fluid from the portable apparatus 220 to remain properly hydrated.

In one embodiment, the processor 226 is operable to execute program instructions that allow the portable apparatus 200 to interact with the user. For example, the processor 226 may include program instructions for enabling operation of the portable apparatus 200 in different modes, such as calibration mode, setting mode, power saving mode, normal operation mode, etc. The program instructions may also enable selective activation and deactivation of the functional modules based on user input(s) received at the control 224. For example, the program instructions may allow the communication module 230 or the display 222 or even the entire apparatus 200 to be selectively turned on and off based on the user input.

In the present embodiment, the portable apparatus 200 further includes a storage or memory module 228 operably connected with the processor 226, the detector 208, and optionally one or more other functional modules. In one example, the memory module 228 is preferably non-volatile memory but may also be volatile memory. In one example, the memory 228 comprises a RAM chip. The memory module 228 may store the one of more of the detected flow properties, present or previous or cumulative liquid consumption data, consumption alarm schedule, as well as their associated time and/or duration.

The detector 208 is arranged to detect flow properties of liquid passing in the channel of the apparatus 200. The detector 208 may be a flow meter arranged to detect flow rate or flow volume of liquid passing in the channel. The flow meter may be a suction-activated flow meter. In one embodiment, the flow meter may include an axial flow turbine arranged in the channel, and a sensor arranged to monitor the movement (e.g., rotation) of the axial flow turbine. The axial flow turbine may be mounted on a shaft and include a plurality of vanes. Preferably, the vanes are angled or spiralled such that the axial flow turbine rotates when liquid flows through it. The turbine and the sensor may magnetically interact with each other, as described with respect to FIGS. 1A-1F. The detector 208 can determine flow rate or flow volume of liquid passing in the channel based on the detected speed of rotation of the turbine or the detected the number of revolutions of the turbine, the time or duration of flow, as well as predetermined geometric parameters of the channel and the turbine.

The portable apparatus 200 further comprises a clock 236 operably connected with the processor. The clock 236 can be used for determining time, and may also be used for determining a time stamp associated with the one or more detected flow properties. This information can be processed by the processor 226, stored in the memory 228, and/or communicated to the external electronic device. Preferably, the clock 236 is arranged to provide the time of occurrence or duration of an event detected by the detector 208 to the processor 226. For example, the clock 236 may associate the liquid consumption event with the time of which it occurred. Furthermore, the clock 236 may help the processor 226 to determine the appropriate time for providing an alarm to the user.

An indicator module 222 is arranged in the portable apparatus 200. The indicator module 222 can be operably coupled with the processor 226 for indicating information relating to liquid consumption. Preferably, the indicator module 222 is operable to indicate time, a cumulative amount of liquid consumed, the amount of liquid consumed in the present or the previous liquid consumption event, percentage representing a ratio of liquid consumed to a targeted liquid consumption, the status of the memory module, the energy capacity remaining in the power source of the portable apparatus, etc. The indicator module 222 may comprise one or more of: a digital display screen e.g., (LED display, OLED display), a light emitting device (e.g., multiple LEDs, fuel gauge), and a sound emitting device (e.g., buzzer).

As shown in FIG. 2, the portable apparatus 200 further comprises control or actuators 224 providing input means for enabling a user to enter input to the processor 226. In one example, the control actuators 224 include push-buttons positioned on the housing near the display 222. In other example, the actuators 224 may include a touch sensitive display screen, one or more button, dial, knob, trigger, etc. A user may use the control actuators to select different operation modes (such as normal measurement mode, setting mode, factory test mode, and calibration mode) of the apparatus 200. A user may further provide input for setting targeted liquid consumption, resetting the time, reviewing consumption history, etc. The control actuators 224 also allow users to easily customise and manage the apparatus 200, for example, by changing a setting of the apparatus 200 (e.g., setting of the brightness of display 222, turning on or off the communication module 230).

The portable apparatus in the present embodiment further includes a communication module 230 arranged to communicate the liquid consumption data (consumption volume, time stamp, etc.) to an external electronic device. Preferably, the communication module 230 is a wireless communication module that enables wireless communication between the apparatus 200 and an external electronic device. Preferably, the communication module 230 may be a Bluetooth communication module that can establish a Bluetooth communication link with the external electronic device. The communication module 230 can also or alternatively include an ANT+ communication module arranged to establish an ANT+ communication link with the external electronic device. Other communication links such as Wi-Fi, ZigBee, NFC, etc., are also possible in other embodiments. The communication between the external electronic device and the communication module 230 of the portable apparatus 200 may be initiated by either the electronic device or the portable apparatus 200. For example, the communication can be established actively by the communication module 230 actively seeking a signal from the external device, or passively by the communication module 230 broadcasting a signal to be picked up by the electronic device. The communication module 230 in the present embodiment is operably connected with the processor 226 and/or the memory module 228 for transferring data gathered by the processor or data stored in the memory module 228 to the external electronic device for storage or further processing or analysis. For example, the data that can be transferred to the external electronic device include the liquid consumption data of the user. In one example, the communication module 230 is also operable to receive data or command from the external electronic device. For example, the communication module 230 may be operable to receive a predetermined liquid consumption alarm from the external electronic device.

The external electronic device operable to communicate with the communication module 230 of the portable apparatus can be any information handling system, preferably a mobile phone, a computer, a smart watch, a tablet, a health tracker device, etc. Preferably, the external electronic device comprises suitable components necessary to receive, store and execute appropriate computer instructions. The components may include one or more of a processing unit, a read-only memory (ROM), a random access memory (RAM), disk drives, input devices such as a power port, a USB port, etc., I/O devices such as a touch sensitive display, a physical or virtual keyboard and communications links. The external electronic device preferably includes instructions that may be included in ROM, RAM or disk drives and may be executed by the processing unit. Optionally, a communication log between the portable apparatus and the electronic device may be recorded in the memory module of the external electronic device.

An inertial measurement unit 232 is arranged in the portable apparatus 200. The inertial measurement unit 232 is operably connected with the processor 226 and arranged to detect an orientation of the portable apparatus 200. In one example, the inertial measurement unit 232 may include an accelerometer, a gyroscope, etc., that can determine an orientation of the portable apparatus 200. Based on the orientation detected by the inertial measurement unit 232, the processor 226 adjusts the orientation of information provided on the display 222 so as to facilitate reading of the information by the user.

The apparatus 200 may further include a power source 234 arranged to power electrical components of the portable apparatus. The power source 234 is operably coupled with all other functional modules in the portable apparatus 200 that need to be powered. In one implementation, the power source 234 may be a rechargeable battery installed in the housing. The battery may be of Lithium based chemistry. The battery may be removable from the housing, or alternately, it may be hard-wired in the apparatus 200. Preferably, a charging port is arranged on the housing of the portable apparatus 200 to allow the rechargeable power source to be charged via a power cable connecting to an external power source (e.g., AC mains). The charging port may be provided with a cover for protecting the port when not in use.

In a preferred embodiment, the power source 234 is operable to cooperate with the processor 226 to selectively operate the functional modules in the portable apparatus 200. For example, the portable apparatus 200 in the present embodiment may be capable to operate in an idle mode when the portable apparatus 200 is not being used (e.g. no rotation of flow turbine is detected by the sensor) and in an active mode when the portable apparatus 200 is being used (e.g. rotation of flow turbine is detected by the sensor). In one embodiment, in the idle mode, only the sensor is in an operation (power on) state for detection, whereas the other functional modules are put in a reduced power (e.g., power off or hibernation) state. On the other hand, in the active mode, the sensor and at least one other functional module as shown in FIG. 2 are in an operation (e.g., power on) state. In other embodiments, the power source 234 can selectively provide power to different functional modules for optimized resource utilization and energy efficiency. For example, the power source 234 may only provide power to the functional modules that need to be operated at that moment, or may dynamically adjust the level of power provided to different functional modules based on the needs or importance of the task to be performed by the different functional modules. By properly controlling the power supplied by the power source 234 to the other functional modules in the portable apparatus 200, energy conservation can be achieved and resource utilization efficient can be improved, thereby prolonging the work time and efficiency of the portable apparatus 200, especially in an outdoor environment where charging source may not be readily available.

Whilst the portable apparatus 200 is shown in the embodiment of FIG. 2 to include nine different functional modules implemented using different mechanical and/or electrical elements, it should be appreciated that the portable apparatus in other embodiments of the invention may omit some functional modules or include more additional functional modules. Structural and functional features of the portable apparatus 100 of FIG. 1 and structural and functional features of the portable apparatus 200 of FIG. 2 can be interchanged, combined, etc.

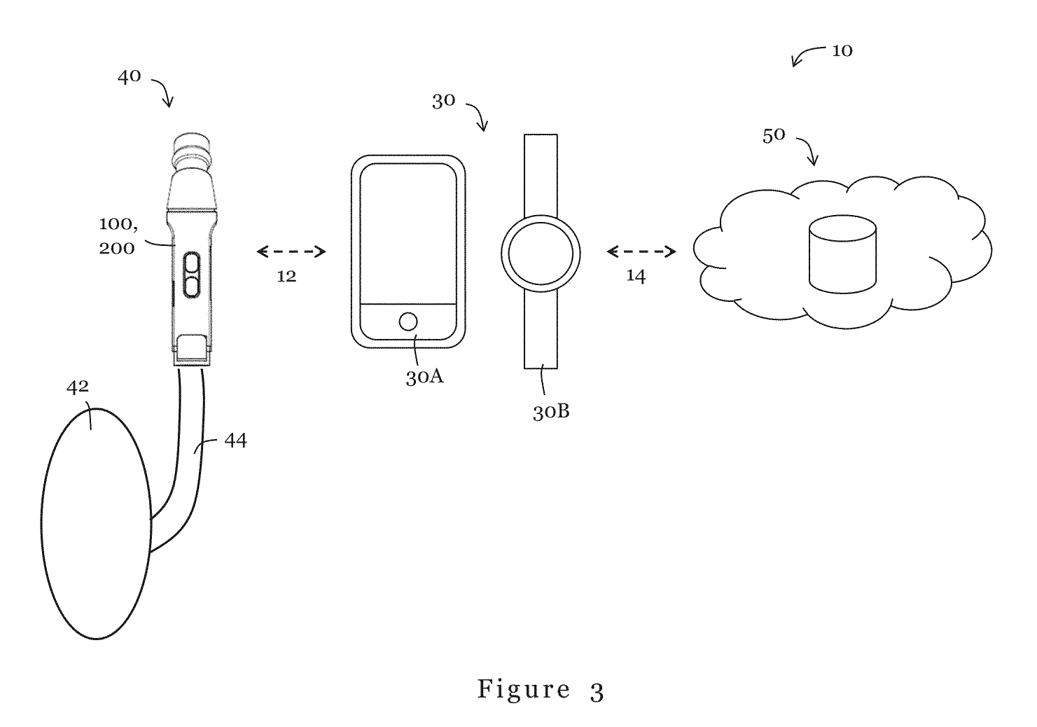

FIG. 3 shows a liquid consumption monitoring system 10 in accordance with one embodiment of the invention. The system 10 includes a portable hydration kit 40 formed by the portable apparatus 100, 200 for monitoring liquid consumption operably connected with a liquid container 42 through a tube 44. In one example, the liquid container 42 may be a hydration bladder. As shown, the portable apparatus 100, 200 is arranged to communicate data or information with an external electronic device 30. In this example, the external electronic device 30 is a smart phone 30A or a smart watch 30B. However, the external electronic device 30 can also be a laptop, a health track device, or other information handling device. Preferably, the external electronic device 30 comprises suitable components necessary to receive, store, and execute appropriate computer instructions. The components may include one or more of a processing unit, a read-only memory (ROM), a random access memory (RAM), disk drives, input devices such as a power port, a USB port, etc., I/O devices such as a touch sensitive display, a physical or virtual keyboard and communications links. The external electronic device preferably includes instructions that may be included in ROM, RAM or disk drives and may be executed by the processing unit.

In the present embodiment, the portable apparatus 100, 200 and the external electronic device 30 are operably connected through a communication link 12 for communicating data. Preferably, the communication link 12 is a wireless communication link, preferably a Bluetooth communication link and/or an ANT+ communication link. In an alternative embodiment, the communication link 12 may be a wired connection between the portable apparatus 100, 200 and the external electronic device 30. In some embodiments, the communication link 12 may be encrypted. In one embodiment, the communication link 12 between the portable apparatus 100, 200 and the external electronic device 30 may be established actively, with the communication module of the portable apparatus 100, 200 actively seeking a signal from the external electronic device 30, or passively, with the portable apparatus 100, 200 broadcasting a signal to be picked up by the electronic device 30. In one embodiment, the communication module of the portable apparatus 100, 200 may be activated by the user through actuating a button of the control actuators on the portable apparatus 100, 200. In another embodiment, the communication module of the portable apparatus 100, 200 may be activated automatically without user intervention.

As shown in FIG. 3, the external electronic device 30 is further connected to a server 50 through another communication link 14. In a preferred embodiment, the external electronic device 30 is connected to a server 50 that is arranged in a cloud computing arrangement or a distributed computing arrangement distributed on individual software, hardware, or a combination of software and hardware components on a computer network, via a communication link 14 which may be encrypted.

In the system 10 of FIG. 3, the portable apparatus 100, 200 is operable to determine flow rate or flow volume of liquid passing in the channel (e.g., consumed by the user) and the associated time or duration. Optionally, the portable apparatus 100, 200 may also determine one or more information relating to a cumulative liquid consumption, the occurrence of a liquid consumption event, a liquid consumption alarm, etc., and their associated time and duration. In one embodiment, the portable apparatus 100, 200 is operable to communicate one or more of these data to the external electronic device 30 via communication link 12, e.g., when the user of the portable apparatus 100, 200 connects the portable apparatus 100, 200 with the external electronic device 30. The data received by the electronic device 30 may be stored locally in a memory module of the electronic device 30 and/or may be further communicated to the server 50 in the cloud via communication link 14. In other implementations, the portable apparatus 100, 200 may be operable to communicate these data automatically (e.g., push data) to the electronic device 30 and optionally to the server 50 in the cloud. In one embodiment, the portable apparatus 100, 200 may be operable to communicate these data automatically to server 50 in the cloud directly without using the electronic device 30. Preferably, the electronic device 30 is operable to retrieve these data from its local memory or from the server 50 in the cloud. The electronic device 30 can display the data to the user through a display screen, provide alarm to the user where appropriate, and/or perform further analysis using the data obtained.

In one embodiment, the user can associate the portable apparatus 100, 200 with the electronic device 30 using an interface at the electronic device. The user may add or remove entries in the event log, measurement data, or edit the liquid consumption received from the portable apparatus 100, 200 using the electronic device 30. The user may also use the data for other fitness related analysis. For example, the user may use the liquid consumption data obtained from the portable apparatus 100, 200 in combination with other fitness related measurements (e.g., food consumption, exercise, etc.) determined by other devices or modules, for health related applications. The user may input into the electronic device 30 a liquid consumption alarm to be transferred to the portable apparatus 100, 200 through the communication link. In one example where the electronic device 30 is a health or fitness related monitoring device (e.g. smart watch) that monitors the physiological signs (such as heart rate, breathing rate, etc.) of the user, the device 30 may determine that the user needs to consume fluid to remain hydrated based on the physiological signs measurements. Accordingly, the device 30 can provide a liquid consumption alarm to the user directly, or can determine and transmit a liquid consumption alarm to the portable apparatus 100, 200 to remind the user of the need to consume fluid.

It should be appreciated that the system in the embodiment of the invention is capable of modifications and variations, and is adapted for operation with different portable hydration kit and electronic devices.

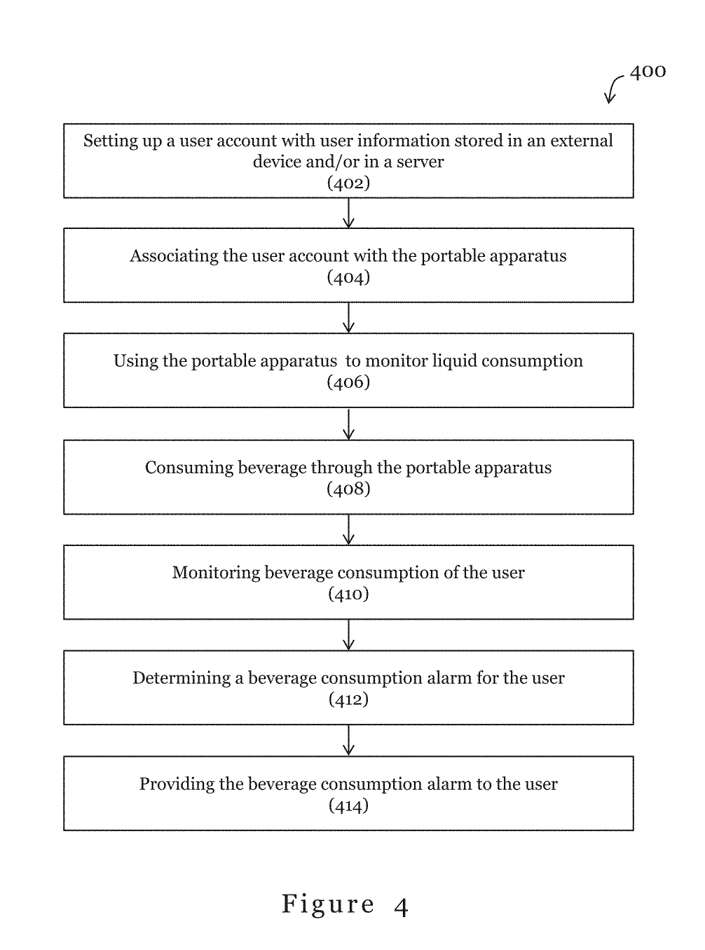

FIG. 4 illustrates the general operation 400 of the liquid consumption monitoring system 10 of FIG. 3 in accordance with one embodiment of the invention. In this embodiment, the method begins from step 402. In step 402, a user of the portable apparatus 100, 200 sets up a user account through a software application or a webpage, using the electronic device 30. The user sets up his account by inputting his personal information such as weight, height, gender, age, etc., as well as the login information such as a username, password, etc., to the application or webpage. The above information may be stored locally in the electronic device 30 and/or in the server 50. In step 404, the user then associates the user account with the portable apparatus 100, 200 for monitoring liquid consumption. The portable apparatus 100, 200 can be associated with the user, for example, by establishing communication between the electronic device 30 and the portable apparatus 100, 200, and transmitting a user-specific token or identifier to the portable apparatus 100, 200. After the portable apparatus 100, 200 has been associated with the user, the user can then use the portable apparatus 100, 200 for consuming liquid beverages, in step 406. The user may carry the portable apparatus 100, 200 for use with a portable liquid source such as a hydration bladder. In step 408, the user may consume beverage through the portable apparatus 100, 200 (e.g., the mouthpiece). In steps 410, the portable apparatus 100, 200 is operable to monitor a volume of liquid consumed by the user. This information may be processed locally in the portable apparatus 100, 200 or may be transmitted to the external electronic device 30. Based on the information detected or determined, and based on the stored data, the portable apparatus 100, 200 or the external electronic device 30 may subsequently generate a beverage consumption alarm for the user to remind the user to consume fluid through the portable apparatus 100, 200, in step 412. In a preferred embodiment, the generation of the beverage consumption alarm also takes into account the weight, height, gender, age of the specific user, in addition to the information determined by the portable apparatus 100, 200 or the electronic device 30. When the communication between the portable apparatus 100, 200 and the server 50 or between the external electronic device 30 and the server 50 is established, the portable apparatus 100, 200 may communicate the beverage consumption alarm, and information associated with the beverage consumption routine of the user to the electronic device 30 and optionally to the server 50. In some cases, however, the information may not be immediately transferred to the electronic device 30 or the server 50. Rather, the information may be stored locally in a memory module of the portable apparatus 100, 200 or electronic device 30, with or without subsequent transfer to the server 50. In step 414, the beverage consumption alarm is broadcasted to the user to remind the user to consume fluid through the portable apparatus 100, 200. In one implementation, the beverage consumption alarm may be provided to the user through either the alarm module of the portable apparatus 100, 200 or through other alarm means in the external device 30, or both.

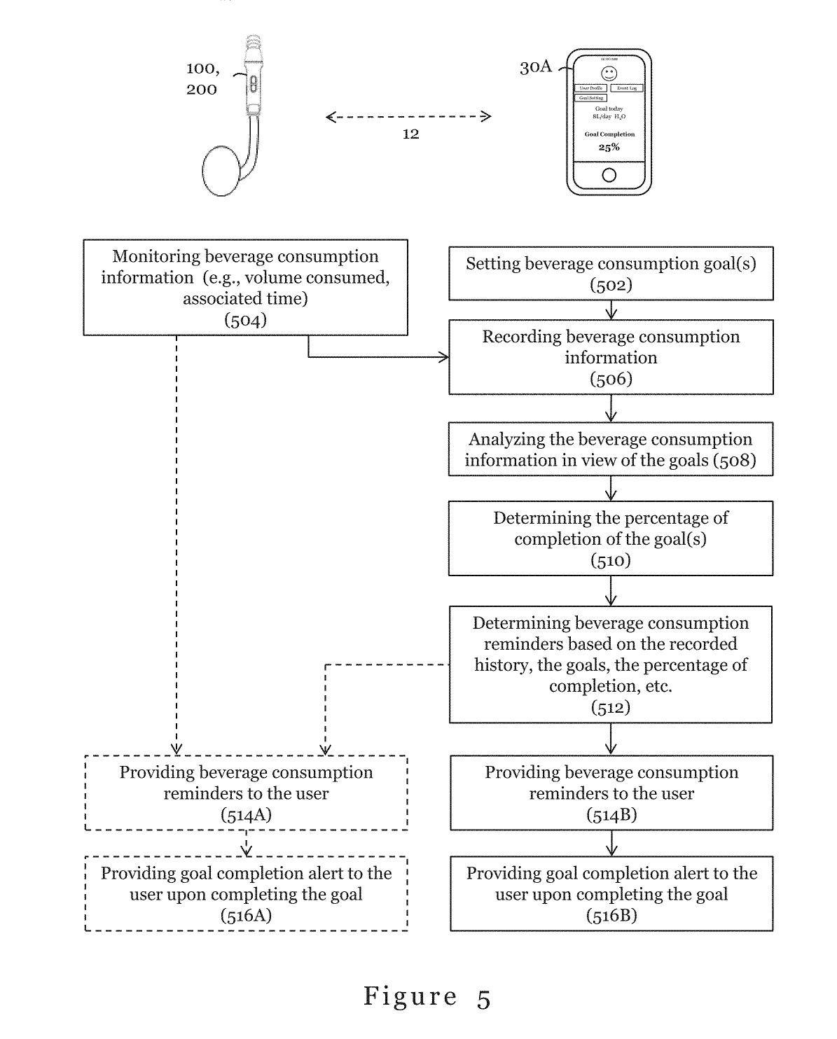

FIG. 5 illustrates an operation of the liquid consumption monitoring system 10 of FIG. 3 in accordance with one embodiment of the invention. In the example of FIG. 5, the portable apparatus 100, 200 is in data communication with the smart phone 30A through a wireless communication link 12. In the present example, the smart phone 30A is installed with a software application (“app”) that allows the user to interact with the portable apparatus 100, 200 (see the exemplary interface on the screen of the phone 30A). Specifically, the software application allows the user to set specific beverage consumption goals such as the amount of beverage (e.g., water) to be consumed in a certain period of time (e.g., per day). After setting the goal, the portable apparatus 100, 200 and/or the smart phone 30A will monitor and analyses the beverage consumption routine of the user, and provide suitable indicators or reminders to the user at appropriate times so as to help to user to achieve the goal.

Referring to FIG. 5, in step 502, the user sets beverage consumption goals through the software application installed on the smart phone 30A. Preferably, the user may set up a user account with his/her personal information (gender, weight, height, etc.), and connect (or “pair”) the portable apparatus 100, 200 with the smart phone 30A through the software application. In one embodiment, the software application is operable determine the amount of beverage (e.g., water) that needs to be consumed by the user for a certain period of time (e.g. daily) based on the user's personal information (gender, weight, height, etc.). In another embodiment, the user may input his or her own beverage consumption goal into the software application, i.e., the amount of beverage (e.g., water) that needs to be consumed by the user for a certain period of time (e.g. daily) is determined by the user him-/her-self. Preferably, the beverage consumption goal, once set in step 502, is stored locally in the smart phone 30A, and optionally, in the server 50. After the initial set up in step 502, in step 504, the portable apparatus 100, 200 detects, monitors, and records the beverage consumption events of the user as previously described. As the user carries out his or her daily activities, he or she may consume beverage through the portable apparatus 100, 200. Accordingly, the portable apparatus 100, 200 may detect the volume of beverage consumed and the associated time or duration.

In step 506, the consumption data of the user as monitored and recorded by the portable apparatus 100, 200 are transmitted to the phone 30A through communication link 12. The communication can be performed periodically, instantaneously, or manually (e.g. by press a sync button on the phone 30A), depending on the availability of the communication link 12 between the portable apparatus 100, 200 and the phone 30A. In step 506, the phone 30A maintains a log of the beverage consumption data including the amount of beverage consumed and the associated time or duration, locally, and optionally, in the server 50. Then, in step 508, the phone 30A analyses the beverage consumption data based on the beverage consumption goal initially set in step 504. The analysis may include calculating the total consumption from the starting time, the time since last consumption, etc.

In step 510, the smart phone 30A determines the percentage of completion of the goal. The phone 30A may display the percentage value on its screen through the interface of the software application. In one example, if the user has initially set a goal of consuming four litres of water for one day, and the beverage consumption data shows that the user has, since the starting time, consumed 1 litres of water, the phone will determine that the goal completion percentage is ¼=25%. The goal completion percentage can be displayed on the electronic device 30A. The phone 30A may also transmit the result to the portable apparatus 100, 200 through the communication link for storage in the local processor or memory of the portable apparatus 100, 200.

After step 510, the phone 30A may then determine beverage consumption reminders or alerts based on the analysis of the beverage consumption data obtained from the portable apparatus 100, 200, in step 512. In particular, the phone 30A may determine beverage consumption reminders based on the event log, the beverage consumption goals set, and/or the percentage of completion of the goal. In one embodiment, the phone 30A may determine the time for alerting the user to consume beverage based on amount of beverage last consumed and/or the time since last consumption to prevent the user from being dehydrated. In addition, the phone 30A may determine the time for reminding the user of the percentage of completion of the goal set initially. The times for providing these alerts to the user is then stored locally in the phone 30A and/or transmitted to the portable apparatus 100, 200 for local storage, through communication link 12. These times are also optionally stored in the server 50.

In steps 514A and 514B, the portable apparatus 100, 200 and/or the phone 30A may provide the alerts to the user at the determined times to remind the user of dehydration, or to remind the user of the outstanding goal set initially. For example, the phone 30A may provide an audible alarm and/or a vibratory alarm that reminds the user the need to consume beverage to stay hydrated. The phone 30A may also display other graphical notifications (e.g., the word “DRINK NOW”, the percentage of goal completed, etc.) on the screen through the interface of the software application, to provide a beverage consumption alert or an outstanding goal alert to the user at the determined time. On the other hand, the portable apparatus 100, 200 may also provide the same alerts to the user using, for example, the alarm module or the display.

In steps 516A and 516B, the portable apparatus 100, 200 and/or the phone 30A may provide the alerts to the user upon determining that the goal is completed. For example, the phone 30A may provide an audible alarm and/or a vibratory alarm, or display other graphical notifications on the screen through the interface of the software application to notify the user that the goal has been completed. The portable apparatus 100, 200 may also provide the same alerts to the user using, for example, the alarm module.

It should be readily appreciated that the software application integration operation steps illustrated in FIG. 5 are merely exemplary. The portable apparatus 100, 200 and the smart phone 30A (or other external electronic device in communication with the beverage portable apparatus) are operable to perform similar functions in a different operation sequence, and are further operable to perform additional functions based on specific implementations. Also, in some embodiments, the external electronic device need not be a smart phone 30A, but can be a tablet, a portable computer, or other type of electronic devices that are operable to communicate wirelessly with the beverage portable apparatus.

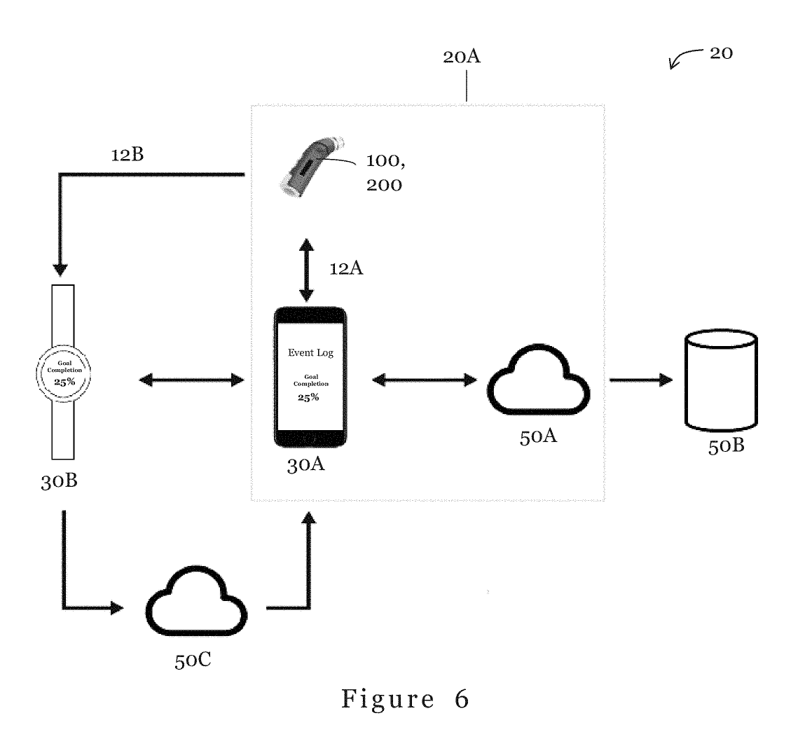

FIG. 6 shows a system 20 for tracking liquid consumption in one embodiment of the invention. The system 20 includes a portable apparatus 100, 200 for monitoring liquid consumption, operably connected with a liquid container through a tube, to form a portable hydration kit. The portable hydration kit may be the portable hydration kit 40 in FIG. 3. The portable apparatus 100, 200 of the portable hydration kit is arranged to determine an amount of liquid consumed by a user from the liquid container. The portable apparatus 100, 200 may further determine other relevant information such as time stamp associated with the liquid consumption. Determined data is then transmitted to an activity tracking device 30B operably connected with the portable apparatus 100, 200 through a communication link 12B. Preferably, the communication link 12B is a direct and wireless communication link which allows the portable apparatus 100, 200 to communicate directly (without going through a server) and wirelessly with the activity tracking device 30B. In one example, the communication link 12B is an ANT+ communication link.

The activity tracking device 30B is arranged to determine an amount of liquid remaining in the liquid container 42 based on the data received from the portable apparatus 100, 200. In this embodiment, the activity tracking device 30B is a smart watch with processor, memory, and wireless communication module. The device 30B may further include a built-in GPS module, one or more physiological measurement modules for determining physiological signals (heart rate, pulse rate, blood oxygen level, etc.) of the user. Preferably, the smart watch stores various activity profiles for activities such as jogging, cycling and hiking. The activity tracker device 30B can also store a data relating to an initial amount of liquid in the liquid container 42. The device 30B can determine the amount of liquid remaining in the liquid container 42 by subtracting the amount of liquid consumed from the known initial amount of liquid inside the liquid container 42.

The activity tracker device 30B is further arranged to determine a liquid consumption plan based on the determined amount of liquid remaining, as well as activity data and user activity data obtained. The activity data may include a type of activity being performed or to be performed by a user and a predetermined duration of the activity being performed or to be performed by the user. These may be inputted at the device 30B by the user. Additionally or alternatively, the activity data may include a completion state of the activity as determined by the device 30B during the activity. Preferably, the activity data includes geographic data and weather condition of the environment in which the activity is being or is to be performed. The geographic data includes latitude, longitude and altitude of the environment, and it may be obtained from the build-in GPS in the activity tracker device 30B before or during the activity. Information of the weather condition may include temperature, humidity, and UV index of the environment. The weather information may be detected by one or more build-in sensors in the activity tracker device 30B or obtained from a server in wireless communication with the smart watch.

In some embodiments, the liquid consumption plan may be determined based also on a liquid consumption record of the user during the activity or a liquid consumption goal set by the user before or during the activity. The liquid consumption plan may be updated as the activity progresses (and as the environmental condition and the physiological conditions of the user change). Accordingly, an existing liquid consumption plan is updated during activity for assisting the user to stay properly hydrated or to reach his liquid consumption goal.

The liquid consumption plan comprises at least one liquid consumption reminder to be provided to the user at a specific time during the activity. The liquid consumption plan may be predetermined based on the activity data inputted by the user prior to the activity, and it may be updated as the activity progresses and the conditions change. The liquid consumption reminder may be provided by the device 30B to the user at determined time points to remind the user to drink a certain amount of liquid within a specific time through the apparatus 100, 200 so as to stay hydrated. The reminder may be in the form of a visual, audio, or tactile alarm. Preferably, the reminder also indicates a specific amount of liquid that the user should consume at a particular time point. For example, during a hike, the activity tracker device 30B may include an initial liquid consumption plan that reminds the user to drink 500 ml of water every hour. But when a steeper ascend is detected by the activity tracker device 30B, the liquid consumption plan will change and the activity tracker device 30B may remind the user to drink more water, e.g. 700 ml of water, every hour during the ascend. All the data determined and detected by the activity tracker device 30B is preferably sent to a server 50C for storage.

The portable apparatus 100, 200 in the system 20 in FIG. 6 is further arranged to transmit the determined data to an electronic device 30A operably connected with the portable apparatus 100, 200 through a wireless communication link 12A, which is further connected to a server. The wireless communication link 12A in this example can be a Bluetooth communication link. This sub-system 20A of the system 20 wherein the portable apparatus 100, 200 is integrated with the electronic device and the server is similar to the system 10 illustrated in FIG. 3.

In addition to transmitting and receiving data to and from the portable apparatus 100, 200, the electronic device 30A is further arranged to receive data from the activity tracking device and/or the server 50C. The received data may include liquid consumption plan, liquid consumption record, activity data and user activity data. Preferably, the electronic device 30A is a mobile phone, a tablet or a computer with a pre-installed software application (“app”) capable of analysing the received data from the activity tracking device 30B and/or portable apparatus 100, 200, and determining a future liquid consumption plan based on the analysed data. For example, the predetermined activity profiles can be adjusted based on the analysed data. The analysed data and liquid consumption plan can be stored in the server 50A, and may further be sent to a third-party database 50B, which may integrate with a social network platform for user to share activity information.

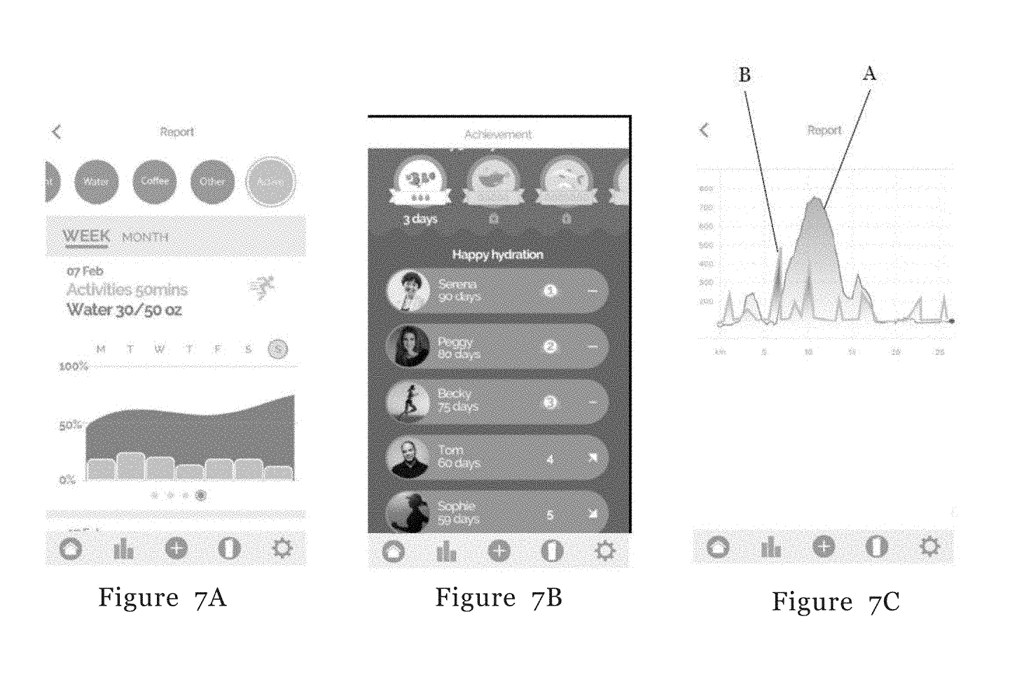

FIG. 7A shows an interface of an activity and liquid consumption record on a software application in one example. In this example, the software application (“app”) is installed on a mobile phone. The mobile phone may be the mobile phone 30A in FIG. 6, which receives data from the portable apparatus and/or the activity tracker device, and further processes the data. The activity record and liquid consumption record of each day in the week can be presented in a graph. The record is shown as an achievement percentage of the actual activity and liquid consumption level to the targeted level pre-set by the user. Both the activity achievement percentage and the liquid consumption achievement percentage are plotted on a single graph, such that the user can easily review his activity and liquid consumption record of the past week at one glance.

FIGS. 7B and 7C show example interfaces of a social network platform for sharing activity information. Preferably, the social network platform can be accessed by general public using their mobile devices. The social network platform can also be accessed by the mobile device 30A in FIG. 6, which may directly communicate with the portable apparatus and/or the activity tracking device for receiving data. Alternatively, the mobile device 30A can access to a server in connection with the portable apparatus and/or the activity tracker device for retrieving data of liquid consumption and activity record. A user can upload the received data to the social network platform for sharing. In one example, the social network platform can display the route taken by the user from the last cycling activity, as illustrated in FIG. 7B. In another example, the platform can display a graph of the elevation (line A) and water consumption (line B) over the whole duration of activity, as illustrated in FIG. 7C. By sharing activity achievements and liquid consumption record on a social network platform, users can motivate each other, share their experience, and meet like-minded people for activity in the future.

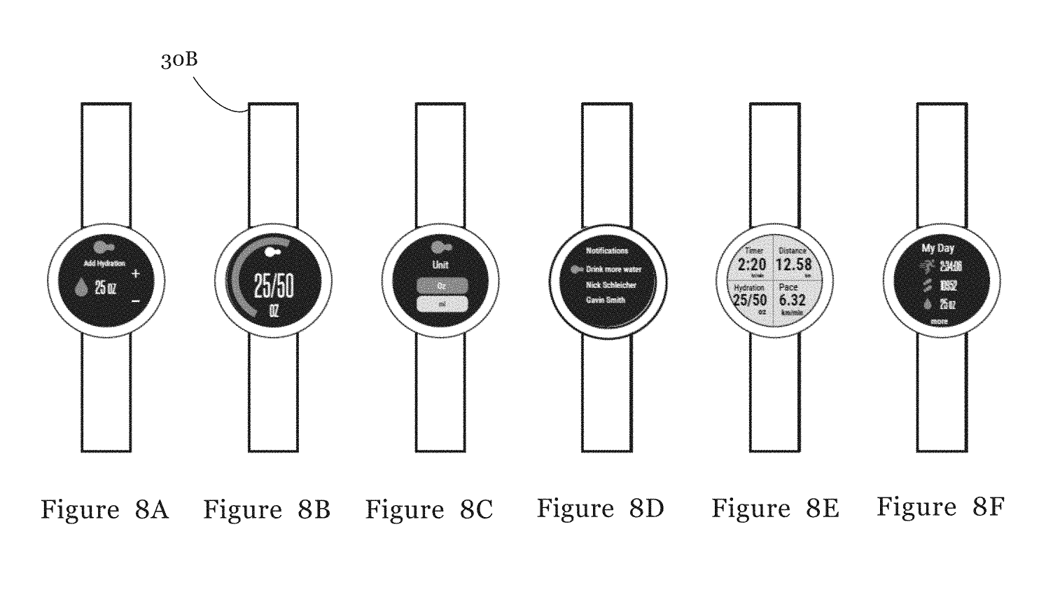

FIG. 8A to FIG. 8F show some example screen interfaces of an activity tracking device 30B. In this embodiment, the activity tracking device 30B is the smart watch of FIG. 6. The smart watch allows the user to set an initial amount of liquid in the liquid container of a portable apparatus in one interface, and displays the initial amount of liquid in another interface. Liquid consumption level is received from the portable apparatus and being processed to show a completion percentage of a liquid consumption goal. The activity tracking device 30B can further provide reminder to user by having notification popping up in one interface. The reminder may include reminding the user to drink more water, informing the user of the risk of dehydration and that water is running out in the liquid container connected with the portable apparatus. In another example interface, the activity tracking device 30B may display useful health and fitness information to the user relating to activity and liquid consumption level, for example but not limited to activity duration, duration and pace of a run, liquid consumption level, number of steps, etc.

Embodiments of the portable apparatus 100, 200 of the invention are advantageous in a number of aspects. For example, the portable apparatus can be readily carried by a user and can automatically monitor fluid consumption. The provision of quick release coupler and the mouthpiece at opposite ends allow quick detachment of the device for assembly and disassembly for charging. The portable apparatus, when used with the external electronic device, is arranged to determine beverage consumption events of the user and hence determine a beverage consumption routine of the user. Alarms can be provided to the user through either the portable apparatus or the external electronic device. This is convenient and particularly useful in outdoor applications where the user may not readily have access to the device or the electronic apparatus to read the information on the display. The portable apparatus in some embodiments of the invention can monitor the daily liquid consumption routine of a user in an automatic and efficient manner. This is partly because of the automatic operation of the apparatus, and the apparatus is capable of energy conservation using an idle mode when appropriate. Furthermore, the portable apparatus is operable to provide alarm automatically to remind the user when and how much fluid needs to be consumed in order for the user to remain properly hydrated. Such alarm can be tailored to the need of the user as the generation of the alarm takes into account not only the beverage consumption record of the user, but also the weight, height, gender, age, and other person information of the user. A log of all beverage consumption activities of the user can be maintained automatically. By providing these functions, the portable apparatus in the invention can integrate seamlessly into the user's activities to improve the health and fitness of the user.

The portable apparatus in the above-embodiments of the invention is further arranged to connect with an activity tracking device and an electronic device to form a system for tracking liquid consumption that is particularly useful during outdoor activities. This liquid consumption tracking system and method are advantageous in that activity and liquid consumption record can be easily processed, stored, displayed, and shared, during activities and afterwards. When integrated with an activity tracking device, useful health and fitness information can be displayed to the user and provide timely reminder according to a liquid consumption plan for assisting user to stay hydrated during activities. When integrated with an electronic device, the user can review the activity and liquid consumption profile during the activity at a glance and keep track of past record.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the invention are either wholly implemented by computing system or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for tracking liquid consumption, comprising:
determining, using a portable apparatus for monitoring liquid consumption, data indicative of an amount of liquid consumed by a user from a liquid container;
transmitting the determined data to an activity tracking device operably connected with the portable apparatus, for determination of at least one of:
an amount of liquid remaining in the liquid container; and
a liquid consumption plan based on an amount of liquid remaining in the liquid container, activity data, and user activity data arranged in the activity tracking device.

2. The method of claim 1, wherein the activity data comprises at least one of:
a type of activity being performed or to be performed by the user;
a predetermined duration of the activity being performed or to be performed by the user;
a completion state of the activity;
geographic data of the environment in which the activity is being or is to be performed; and
weather condition of an environment in which the activity is being or is to be performed.

3. The method of claim 2, wherein the weather condition comprises at least one of:
humidity level of the environment;
temperature level of the environment; and
UV index of the environment.

4. The method of claim 2, wherein the geographic data includes at least one of: latitude, longitude and altitude of the environment.

5. The method of claim 1, wherein the user activity data comprises physiological data of the user during the activity.

6. The method of claim 5, wherein the physiological data includes at least one of: heart rate, pulse rate, and blood oxygen level of the user.

7. The method of claim 1, wherein the liquid consumption plan is determined further based on a liquid consumption record of the user during the activity.

8. The method of claim 1, wherein determination of a liquid consumption plan comprises updating an existing liquid consumption plan.

9. The method of claim 1, wherein the liquid consumption plan is determined further based on a liquid consumption goal set by the user.

10. The method of claim 1, further comprising:
storing, at the activity tracking device, data of an initial amount of liquid in the liquid container.

11. The method of claim 1, wherein the liquid consumption plan comprises at least one liquid consumption reminder to be provided to the user at a specific time.

12. The method of claim 11, wherein the liquid consumption reminder comprises an amount of liquid to be consumed.

13. The method of claim 1, further comprising:
determining, at the activity tracking device, at least one of:
an amount of liquid remaining in the liquid container; and
a liquid consumption plan based on an amount of liquid remaining in the liquid container, activity data, and user activity data arranged in the activity tracking device.

14. The method of claim 1, wherein the portable apparatus is arranged to communicate directly and wirelessly with the activity tracking device.

15. The method of claim 1, further comprising:
transmitting the determined data to an electronic device operably connected with the portable apparatus for record or analysis.

16. The method of claim 15, wherein the electronic device is arranged to receive, from the activity tracking device, at least one of: liquid consumption plan, liquid consumption record, activity data, and user activity data.

17. The method of claim 15, wherein the electronic device is arranged to analyze data received from the activity tracking device and from the portable apparatus.

18. The method of claim 18, wherein the electronic device is arranged to use the analysed data for determination of future liquid consumption plan.

19. The method of claim 15, wherein the electronic device comprises a mobile phone, a tablet, or a computer.

20. The method of claim 1, wherein the portable apparatus comprises:
an inlet arranged to receive liquid;
an outlet arranged to output the received liquid;
a channel enabling fluid communication between the inlet and the outlet;
a detector arranged to detect one or more flow properties of liquid passing in the channel for determination of an amount of liquid consumed by a user from a liquid container; and
a communication module operably connected with the detector for communicating data indicative of the amount of liquid consumed to an external electronic device.

21. A system for tracking liquid consumption, comprising:
a portable apparatus for monitoring liquid consumption, arranged to
determine an amount of liquid consumed by a user from a liquid container;
transmit the determined data to an activity tracking device operably connected with the portable apparatus, for determination of at least one of:
an amount of liquid remaining in the liquid container; and
a liquid consumption plan based on an amount of liquid remaining in the liquid container, activity data, and user activity data arranged in the activity tracking device.

22. A method for tracking liquid consumption, comprising:
receiving, from a portable apparatus for monitoring liquid consumption from a liquid container, data indicative of an amount of liquid consumed by a user from the liquid container; and
determining, based on the data received, an amount of liquid remaining in the liquid container.

23. The method of claim 22, further comprising:
determining, based on the received data, activity data, and user activity data, a liquid consumption plan.

24. The method of claim 23, wherein the activity data comprises at least one of:
a type of activity being performed or to be performed by the user;
a predetermined duration of the activity being performed or to be performed by the user;
a completion state of the activity;
geographic data of the environment in which the activity is being or is to be performed; and
weather condition of an environment in which the activity is being or is to be performed.

25. The method of claim 23, wherein the user activity data comprises physiological data of the user during the activity.

26. The method of claim 25, wherein the physiological data includes at least one of: heart rate, pulse rate, and blood oxygen level of the user.

27. The method of claim 23, wherein the liquid consumption plan is determined further based on a liquid consumption record of the user during an activity.

28. The method of claim 23, wherein determination of a liquid consumption plan comprises updating an existing liquid consumption plan.

29. The method of claim 23, wherein the liquid consumption plan is determined further based on a liquid consumption goal set by the user.

30. The method of claim 22, further comprising:
storing data of an initial amount of liquid in the liquid container.

31. The method of claim 23, wherein the liquid consumption plan comprises at least one liquid consumption reminder to be provided to the user at a specific time.

32. The method of claim 31, wherein the liquid consumption reminder comprises an amount of liquid to be consumed.
